(19) United States
(12) Patent Application Publication
(10) Pub. No.: US 2004/0204649 A1
(43) Pub. Date: Oct. 14, 2004

(54) Method and system for migrating presets between medical diagnostic imaging system platforms

(76) Inventors: Sankaralingam Ramraj, San Jose, CA (US); Scott Luan, Palo Alto, CA (US)

Correspondence Address: Attn: Elsa Keller, Legal Administrator, Siemens Corporation, Intellectual Property Department, 170 Wood Avenue South, Iselin, NJ, 08830, US

(21) Appl. No.: 10/411,630

(22) Filed: Apr. 11, 2003

(51) Int. Cl. A61B 8/00

(52) U.S. Cl. 600/437

(57) ABSTRACT

A method for migrating presets between medical diagnostic imaging system platforms is presented in which a preset for a first medical diagnostic imaging system platform is provided to a preset translator. The preset translator translates the preset to a second medical diagnostic imaging system platform, and the translated preset is implemented on a medical diagnostic imaging system comprising the second medical diagnostic imaging system platform. A system and method for sharing medical diagnostic imaging system presets are also presented. The system comprises a server and first and second medical diagnostic imaging systems in communication with the server. A medical diagnostic imaging system preset is sent from the first medical diagnostic imaging system to the server, and the preset is stored in the server. A request for the medical diagnostic imaging system preset is sent to the server, and the requested medical diagnostic imaging system preset is sent from the server to the second medical diagnostic imaging system.

### BACKGROUND

A preset is a stored collection of values for one or more parameters of a medical diagnostic ultrasound imaging system (or other type of medical diagnostic imaging system). A user can select a preset to automatically configure an ultrasound system to make workflow more efficient. In some situations, when a software upgrade is performed on an ultrasound system, previously-stored presets are lost and must be recreated by a sonographer. Because sonographers often create a large number of presets, recreating those presets can be a time-consuming and cumbersome process. To avoid recreating stored presets after a software upgrade, a sonographer can copy the presets onto removable media before the upgrade and restore them on the same ultrasound system after the upgrade is complete. However, when an ultrasound system becomes obsolete and is replaced by a new ultrasound system with a different platform, the presets stored on the old ultrasound system cannot be ported or migrated to the new ultrasound system platform, and the sonographer must manually recreate the presets.

There is a need, therefore, for a method and system for migrating presets between medical diagnostic imaging system platforms.

### SUMMARY

The present invention is defined by the following claims, and nothing in this section should be taken as a limitation on those claims.

By way of introduction, in one embodiment, a method for migrating presets between medical diagnostic imaging system platforms is presented. In this method, a preset for a first medical diagnostic imaging system platform is provided to a preset translator. The preset translator translates the preset to a second medical diagnostic imaging system platform, and the translated preset is implemented on a medical diagnostic imaging system comprising the second medical diagnostic imaging system platform. In another embodiment, a system and method for sharing medical diagnostic imaging system presets are presented. The system comprises a server and first and second medical diagnostic imaging systems in communication with the server. A medical diagnostic imaging system preset is sent from the first medical diagnostic imaging system to the server, and the preset is stored in the server. A request for the medical diagnostic imaging system preset is sent to the server, and the requested medical diagnostic imaging system preset is sent from the server to the second medical diagnostic imaging system. Other embodiments are provided, and each of the embodiments described herein can be used alone or in combination with one another.

The embodiments will now be described with reference to the attached drawings.

### BRIEF DESCRIPTION OF THE DRAWINGS

FIG. 1 is a block diagram of a medical diagnostic ultrasound imaging system of an embodiment.

FIG. 2 is a block diagram of an embodiment in which a preset translator is located in a server.

FIG. 3 is a block diagram of an embodiment in which a preset translator is located in a source ultrasound system.

FIG. 4 is a block diagram of an embodiment in which a preset translator is located in a destination ultrasound system.

FIG. 5 is a flow chart of a method of an embodiment for translating a preset between ultrasound system platforms.

FIG. 6 is a block diagram of an embodiment for sharing ultrasound system presets.

FIG. 7 is a block diagram of an embodiment in which a preset in a common format is sent from a first ultrasound system to a server.

FIG. 8 is a block diagram of an embodiment in which a preset in a common format is sent from a first ultrasound system to a second ultrasound system.

FIG. 9 is a block diagram of an embodiment in which a preset in a common format is sent from a first ultrasound system to a server, which translates the preset.

FIG. 10 is a block diagram of an embodiment in which a preset in a common format is sent from a server to a second ultrasound system.

### DETAILED DESCRIPTION OF THE PRESENTLY PREFERRED EMBODIMENTS

Turning now to the drawings, FIG. 1 is a block diagram of a medical diagnostic imaging system of an embodiment. In this embodiment, the medical diagnostic imaging system takes the form of a medical diagnostic ultrasound imaging system 100. While an ultrasound system is used to illustrate this embodiment, it should be noted that other types of medical diagnostic imaging systems can be used. As shown in FIG. 1, the ultrasound system 100 comprises a transducer probe 105, a beamformer 110, a processor 120, a display device 130, a storage device 140, and a user interface 150. The term “processor” broadly refers to the appropriate hardware component(s) of the ultrasound system 100 that can be used to implement the functionality described below. The storage device 140 (or another storage device) stores software (i.e., computer-readable program code) run by the processor 120. The ultrasound system 100 can comprise additional components, which are not shown in FIG. 1 for simplicity.

During an ultrasound examination, a sonographer contacts the transducer probe 105 with a patient, and the ultrasound system 100 generates an ultrasound image. In general, the ultrasound system's processor 120 causes the beamformer 110 to apply a voltage to the transducer 105 to cause it to vibrate and emit an ultrasonic beam into the portion of the patient's body in contact with the transducer 105. Ultrasonic energy reflected from the patient's body impinges on the transducer 105, and the resulting voltages created by the transducer 105 are received by the beamformer 110. The processor 120 processes the sensed voltages to create an ultrasound image and displays the image on the display device 130. In addition to being displayed on the display device 130, a generated ultrasound image can also be stored in digital form in the storage device 140 for later review. Images can also be transferred to removable media (e.g., a magneto-optical disk) or sent over a network (e.g., a local area network in a hospital or the Internet).

The ultrasound system 100 has a number of parameters that can be adjusted for a given examination, such as those that are set prior to the start of the imaging session to optimize image quality. While a sonographer can use the user interface 150 to select a set of parameters from scratch each time he uses the ultrasound system 100, a sonographer will often merely select a stored preset to automatically configure the ultrasound system 100. As used herein, a “preset” is a stored collection of values for one or more parameters of an ultrasound system. (The terms “item” and “parameter” will be used interchangeably herein.) Examples of such parameters include system parameters (such as screen resolution and annotation fonts) and system configuration parameters (such as how much memory should be used for on-board clip review and image transfer over a network). Parameters can also include application settings or imaging parameters such as annotation level (e.g., no annotation, text without image, both text and image), which key activates an auto freeze function, default playback speed (e.g., if two image clips are being played, the playback delay of one clip with respect to the other), ECG display option (e.g., no ECG, ECG without image, both ECG and image, position of ECG), clip review position (e.g., which one of four quadrants to display a clip), number of stages, protocol type (e.g., pulse wave mode), printer selection, clip size, auto review, compression ratio, imaging mode, power, gain, depth, strip size, power steer angle, Automatic Gain algorithm type, black/white inversion mode (e.g., black or white background), image focus position, frequency, image size, left/right inversion, up/right inversion, body marker, pan box, cursor, calc (measurement) settings, physical I/0 settings, right/left foot switch settings, 3D application settings, text annotation settings, contrast agent settings, B-Mode settings, color Doppler mode settings, Spectral mode settings, and M-Mode settings. It should be noted that the foregoing list of examples is not meant to be exhaustive and that other parameters can be used. Further, a preset can include one or more than one parameter, and the ultrasound system 100 can have one or more than one preset. Additionally, a preset can be user-defined or defined by a manufacturer of the ultrasound system.

Depending on the parameters involved, a preset may be specific to a particular ultrasound system platform. An “ultrasound system platform” refers to the hardware (e.g., the processor) and/or software components (e.g., the operating system) that are used by the ultrasound system. Different platforms can exist between different ultrasound systems manufactured by the same manufacturer or between different ultrasound systems manufactured by different manufacturers. A problem can occur when a user wishes to use a preset for one ultrasound system platform on another ultrasound system platform. For example, when a sonographer switches from an obsolete ultrasound system platform to a new ultrasound system platform, the presets for the obsolete ultrasound system platform currently cannot be ported or migrated to the new ultrasound system platform. As a result, the sonographer has to perform the time-consuming and cumbersome task of manually recreating the presets on the new ultrasound system platform.

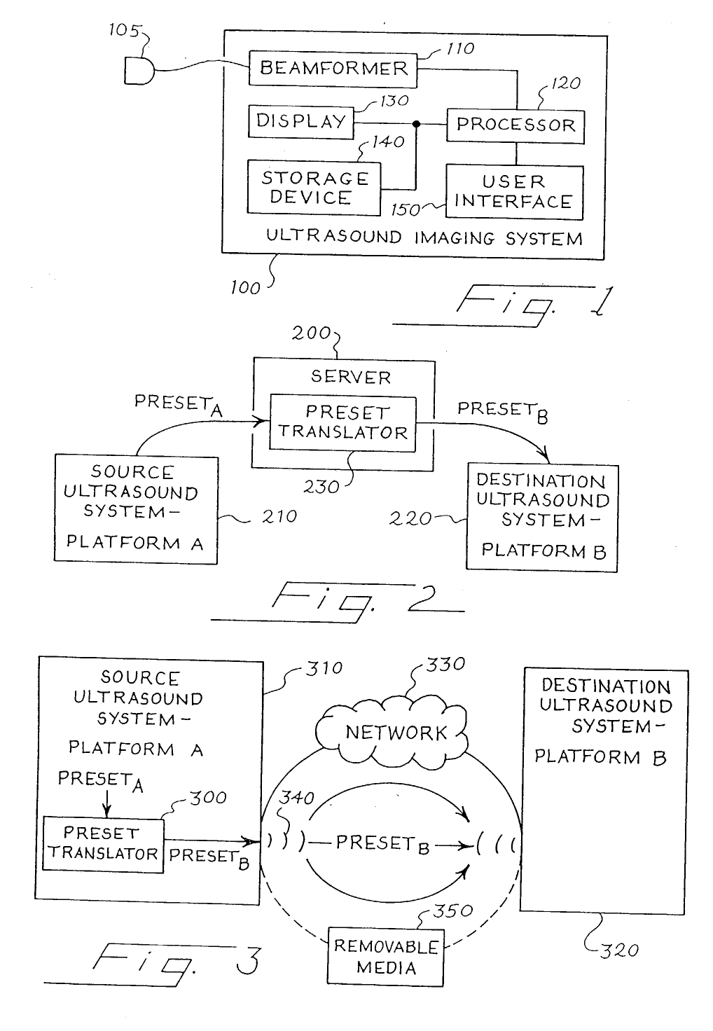

FIG. 2 is a block diagram of an embodiment that can be used to overcome this problem. As shown in FIG. 2, a server 200 connects two ultrasound systems comprising different platforms—a source ultrasound system 210 comprising platform A and a destination ultrasound system 220 comprising platform B. The terms “source” and “destination” are used to designate which ultrasound system serves as the source and destination of a particular preset. The source for one preset can be the destination of another preset. Although shown as connecting only two ultrasound systems, the server 200 can be accessible to other machines, such as, for example, if the server 200 were part of a hospital network or accessible over the Internet. In this embodiment, the server 200 comprises a preset translator 230 that translates a preset specific to platform A to a preset that can operate on platform B. As used herein, the term “server” refers to hardware and/or software components that can be used to implement the functionality described herein attributed to the server (e.g., processing the preset conversion and communicating with modality systems). A server can be, for example, a network computer, a wireless laptop, a personal digital assistant, or even another ultrasound system. In this embodiment, the preset translator is software run on the server.

In operation, the preset specific to platform A (Preset_{A}) is sent from the source ultrasound system 210 to the server 200. The preset translator 230 translates the preset, and the translated preset (Preset_{B}) is sent from the server 200 to the destination ultrasound system 220. The translated preset is then implemented on the destination ultrasound system 220 (i.e., the parameters of the ultrasound system 220 that are identified in the preset are configured in accordance with the values of the parameters specified in the preset). As shown in this embodiment, the use of a preset translator to translate a preset from one platform to another provides seamless migration or porting of presets, and eliminates the need for a sonographer to worry about incompatibilities between different platforms (e.g., different hardware and/or software releases).

In this embodiment, ultrasound system platforms A and B are on two separate ultrasound systems. However, this embodiment can also be applied to the situation where ultrasound system platforms A and B are on the same ultrasound system. For example, an ultrasound system may be using platform A before a system upgrade and platform B after the system upgrade. In this situation, a user can upload the preset specific to platform A to the server 200 before the upgrade and download the translated preset from the server 200 after the upgrade to platform B is complete.

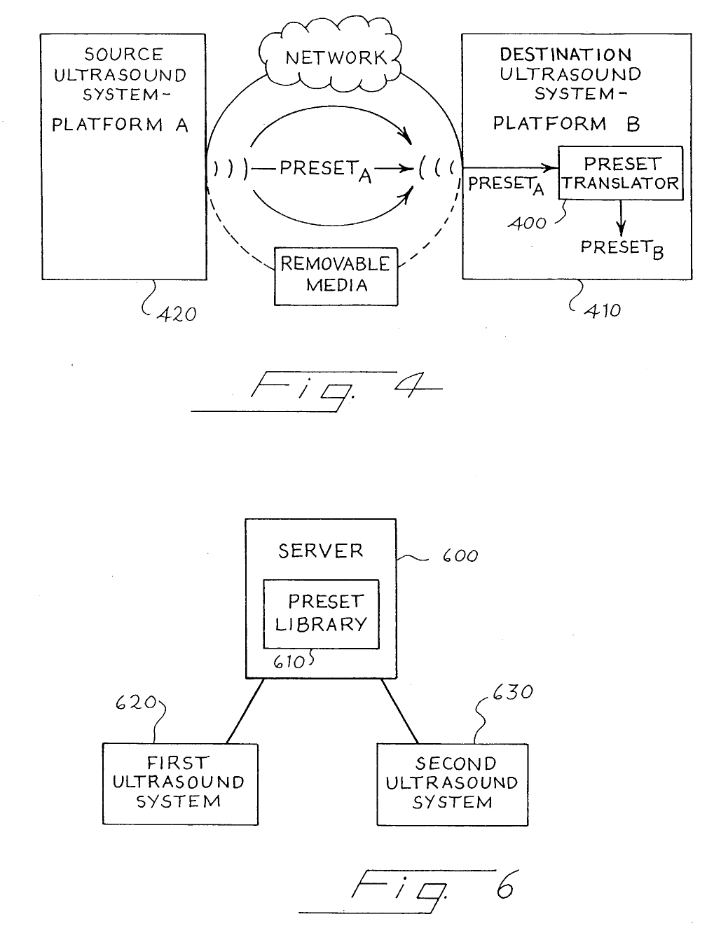

FIGS. 3 and 4 are additional embodiments that can be used to provide preset migration between ultrasound system platforms. In FIG. 3, instead of being in a server, the preset translator 300 is part of the source ultrasound system 310. Accordingly, the translation from Preset_{A} to Preset_{B} occurs at the source ultrasound system 310. The translated preset is made available to the destination ultrasound system 320 by any appropriate mechanism, such as a network 330 (e.g., a local area network, a wide area network, the Internet, etc.), a wireless transmission 340, or removable media 350 (e.g., a magneto-optical disk, a memory card, a laptop computer, a personal digital assistant (PDA), a digital camera, etc.). Additionally, one or more of these mechanisms can be used in combination. For example, the source ultrasound system 310 can wirelessly transmit the translated preset to a PDA (wireless transmissions from an ultrasound system to a PDA are described in U.S. Pat. No. 6,440,072, which is hereby incorporated by reference), which can then send the preset over the Internet via an email to a laptop computer, which can then store the preset in a memory card that is later removed from the laptop computer and placed into the destination ultrasound system 320.

In FIG. 4, the preset translator 400 is part of the destination ultrasound system 410 instead of the source ultrasound system 420. In this embodiment, the un-translated preset (Preset_{A}) is made available to the destination ultrasound system 410 by any appropriate mechanism, and the destination ultrasound system 410 performs the translation. The embodiment shown in FIG. 4 may be preferred when there is more than one possible destination ultrasound system platform. For example, if the preset translator were in the source ultrasound system in such a situation, the user requesting the translation may need to provide the preset translator with information identifying which destination ultrasound system platform is desired. By having the preset translator 400 be part of the destination ultrasound system 410, the preset translator 400 knows which destination ultrasound system platform is desired.

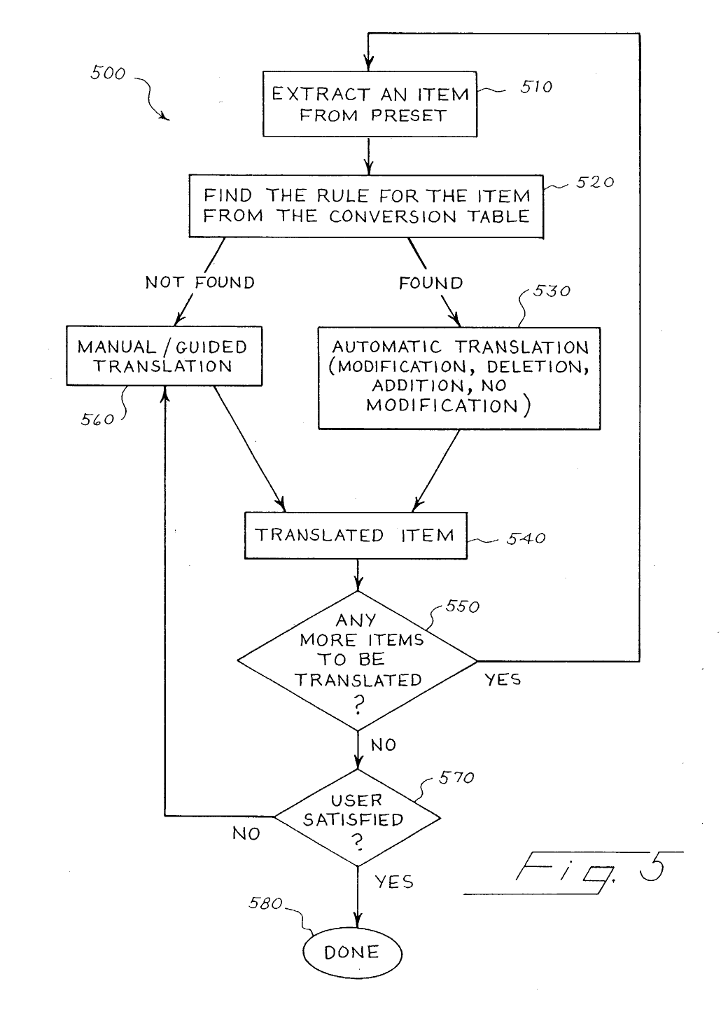

In each of these embodiments, the preset translator can take any desired form. For example, preset translator can be software executed by a general-purpose processor or can take the form of a customized hardware component. Further, the preset translator can use any appropriate method to translate the preset. FIG. 5 is a flow chart 500 showing a method of one embodiment for translating a preset between ultrasound system platforms. In this method, a conversion table is used to translate the preset. It is important to note that other methods (including those that do not use a conversion table) can be used. To illustrate the operation of this method, consider an example in which a preset containing the following four parameters (or items) is to be translated from platform A to platform B: screen resolution, annotation font, automatic gain algorithm type, and specialized item A. The values for this preset for two ultrasound system platforms (platforms A and B) are shown below:

| | Platform A | Platform B |
|---|---|---|
| Screen Resolution | 764 × 1028 | 640 × 580 |
| Annotation Font | Times New Roman | Times New Roman |
| Automatic Gain Algorithm | Available | Unavailable |
| Type | | |

| | |
|---|---|
| Specialized Item A | (No conversion rule available) |

Turning now to the flow chart 500, an item is extract from the preset (act 510). Here, the screen resolution item is extracted. Next, the method attempts to find a rule for this item in the conversion table (item 520). In this example, a rule is found in the conversion table, and an automatic translation is performed (act 530). For this item, the automatic translation maps the 764×1028 image size of platform A to the 640×580 image size of platform B to provide a translated item (act 540). Next, it is determined if there are any more items in the preset to be translated (act 550). In this example, there are three more items to be translated, so the method returns to act 510, and an attempt is made to translate the annotation font. For this item, no modification is needed since the value of the item (Times New Roman) is the same for both platforms. When an attempt is made to translate the automatic gain algorithm type, the conversion table states that this item is unavailable on platform B. As a result, the item is deleted. If the conversion was being made from platform B to platform A, the automatic gain algorithm type item can be added to the preset made available to platform A even though it was not available on platform B. For example, the conversion table can include a rule that generates a value for the automatic gain algorithm type item based on gain and other settings of the source ultrasound system.

When an attempt is made to translate the last item (specialized item A), the method determines that a rule for this item cannot be found in the conversion table. In this situation, the method performs a manual/guided translation technique (act 560) to allow a user to (manually or via prompts from the ultrasound system) input information in an attempt to generate a value for the unknown item. It should be noted that, even if a conversion rule is available, the translation may not occur depending on the feature, application, and/or system settings on the destination ultrasound system. For example, there may be a conversion rule relating to the color that should be used to represent blood flow in a 3D color Doppler image. However, if the destination ultrasound system is not using a color Doppler application, the translation need not occur.

If there are no more items to be translated, the translated items are presented to the user for review to determine if the user is satisfied with the translations (act 570). In some situations, the translation may change the value of an item that the user was satisfied with. For example, a translation may change the value of gain from a desired 30 dB to an undesired −20 dB. Providing the opportunity for review allows the user to request manual/guided translation for any item for which he is not satisfied with the translation (act 560). Otherwise, the translation is complete (act 580).

Turning again to the drawings, FIG. 6 is a block diagram of a system for sharing ultrasound system presets. As shown in FIG. 6, a server 600 comprising a storage device storing a preset library 610 is in communication with first and second ultrasound systems 620, 630. Although shown as being directly connected to the server 600, the ultrasound systems 620, 630 can be connected to the server 600 via a network. In operation, the first ultrasound system 620 sends an ultrasound system preset to the server 600, which stores it in the preset library 610. The preset library 610 can contain other presets sent to it by the same or other ultrasound systems or that are provided to the server 600 in some other fashion (e.g., via a magneto-optical disk). Preferably, presets stored on the server 600 are categorized and searchable by criterion such as, but not limited to, sonographer/physician name (e.g., Dr. Bocanegra, Dr. Altekruse), patient name (e.g., Karen Klafeta, Nicole Pconka, Tracy Sloan), type of examination (e.g., general radiology, vascular examination, cardiology), patient-dependent physical parameters (e.g., weight, sex), and ultrasound system platform. If a user of the second ultrasound system 630 wants to access a preset stored on the server, the user sends a request for that preset to the server 600, which responds by sending the requested preset to the second ultrasound system 630. In this embodiment, the selection of a preset if performed by a user on the second ultrasound system 630 (the destination ultrasound system). In an alternate embodiment, the user uses a separate device (such as a personal computer, a telephone, a PDA, another ultrasound system, etc.) to search the stored presets and/or to request a stored preset be sent to the second ultrasound system 630.

With this embodiment, a user of the first ultrasound system 620 can use the server 600 as a preset repository from which other users can conveniently access stored presets, thereby avoiding repeating the first user's efforts in manually configuring the presets. In this way, the server 600 provides sonographers a medium to share presets. A business service can be provided using this embodiment in which a user who accesses stored presets is charged for the service, and the user providing the stored preset (and/or the owner of the server) receives all or a portion of that charge.

In the above example, the user of the second ultrasound system 630 is a different person from the user who established the preset. However, this embodiment can also be used to make a preset available to a user when that user is using a different ultrasound system. Consider, for example, the situation in which the first ultrasound system 620 is the user's primary system, and the second ultrasound system 630 is located at the user's secondary office. With this embodiment, the user can store the presets used on the first ultrasound system 620 in the server 600 so that he can access those presets when he uses the second ultrasound system 630 in his secondary office. In this way, the user can use the server 600 to access presets that were previously used to scan a particular patient in the primary office when the user examines the patient in the secondary office.

It should be noted that the server 600 need not contain a preset translator. If the stored preset is compatible with the platform of the destination ultrasound system, a preset translator may not be needed at all. However, if a preset translator is desired (e.g., when the requested preset is incompatible with the platform of the destination ultrasound system), the preset translator can be located in any suitable location, such as, for example, in the server 600 or in one of the ultrasound systems 620, 630.

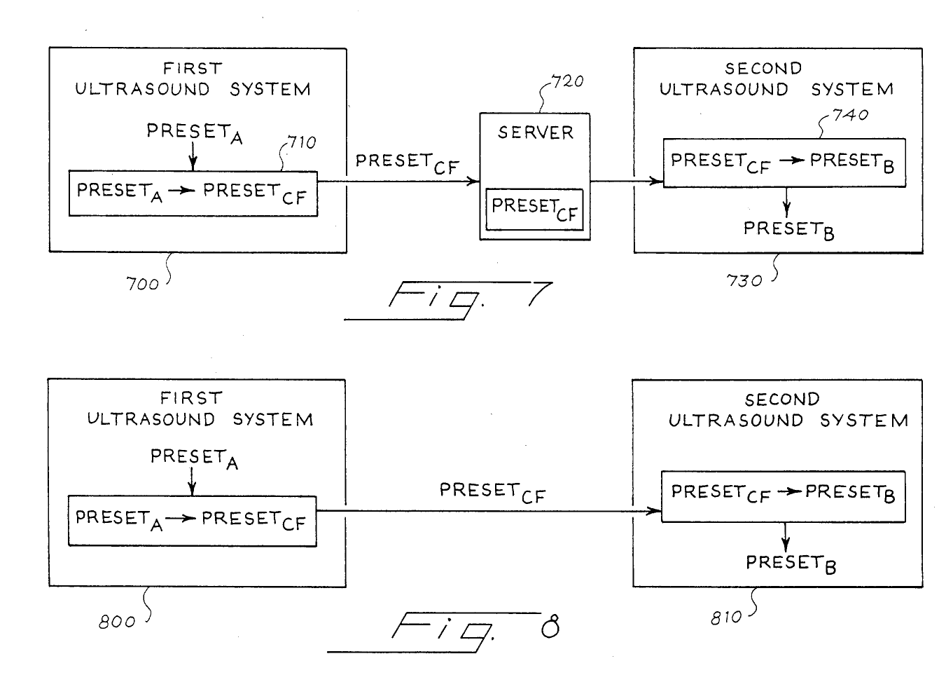

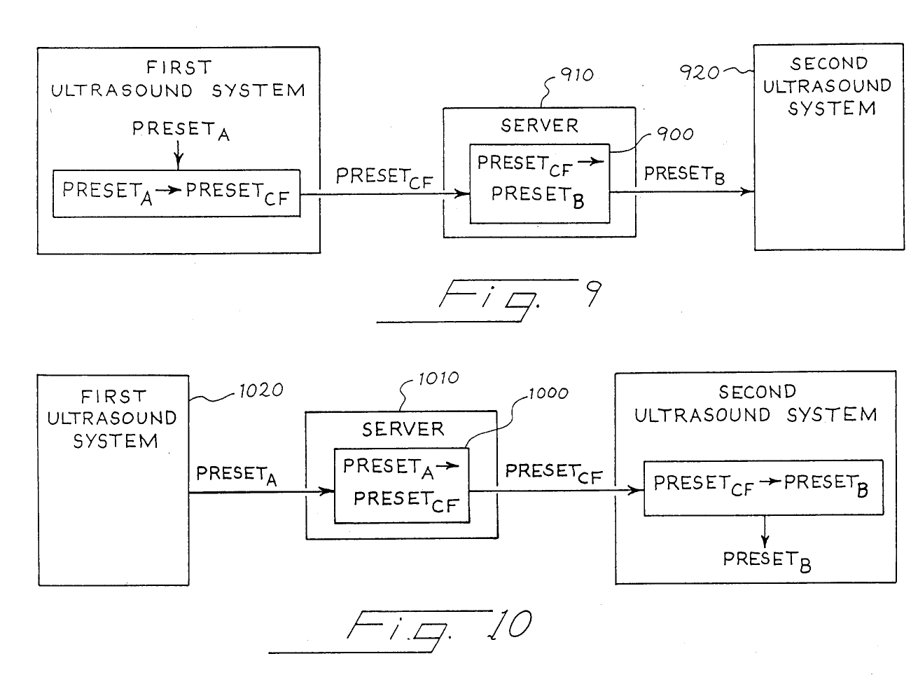

It should also be noted that an intermediate and common format can be used in the translation mechanism so that one component is not required to know about the preset format of the other component. FIGS. 7-10 will now be discussed to illustrate this alternative. In FIG. 7, a first ultrasound system 700 comprises a translator 710 to translate a preset from a first ultrasound system platform (Preset_{A}) to a common format (Preset_{CF}). Preset_{CF} is then sent from the first ultrasound system 700 to a server 720, where it is stored until it is sent to a second ultrasound system 730. The second ultrasound system 730 comprises a preset translator 740 that translates Preset_{CF} to the second ultrasound system platform (Preset_{B}). FIG. 8 is similar to FIG. 7, but Preset_{CF} is sent from a first ultrasound system 800 to a second ultrasound system 810 without the use of a server. In FIG. 9, the preset translator 900 is located in the server 910 instead of the second ultrasound system 920. The embodiment in FIG. 10 has the translator 1000 that translates the preset from the first ultrasound system platform (Preset_{A}) to the common format (Preset_{CF}) located in the server 1010 instead of in the first ultrasound system 1020.

The following is pseudo code for one presently preferred embodiment:

| | | |
|---|---|---|
| 1. | Find the source system from where the preset is copied | |
| | from. | |
| | $source=systemId($preset) | (Code 1) |
| 2. | Find the destination system to where the preset is | |
| | copied to. | |
| | $destination=systemId($destination_preset) | (Code 2) |
| 3. | Is the conversion possible? | |
| | $translation=isConversionAllowed($source, $destination) | (Code 3) |
| 4. | If yes, proceed to step 5; otherwise stop. | |
| | if(!$translation) stop | (Code 4) |
| 5. | Read the item from the preset entries. | |
| | $item=extract($preset) | (Code 5) |
| 6. | Parse the item to the item name (entry) and value. | |
| | $entry=name($item) | (Code 6) |
| | $value=value($item) | (Code 7) |

| | |
|---|---|
| 7. | Look for the entry in the conversion table. If found, apply the rule; |

otherwise, ask the user to translate.

| | | |
|---|---|---|
| | $conversion=lookup($entry, $translation_table) | (Code 8) |
| | if($conversion) $new_value=$conversion($value) | (Code 9) |
| | else $new_value=askUser($entry, $value) | (Code 10) |
| | archive($entry, $new_value) | (Code 11) |
| 8. | Find any remaining item. | |
| | $end=end_of_item($preset) | (Code 12) |
| | if($end) go to step 9 | (Code 13) |
| | else go to step 1 | (Code 14) |
| 9. | Deposit the preset. | |
| | deposit($preset) | (Code 15) |

Finally, as noted above, although an ultrasound imaging system was used to illustrate these embodiments, other types of medical diagnostic imaging systems can be used, such as, for example, those that use any of the following imaging modalities: computed tomography (CT), magnetic resonance imaging (MRI), computed radiography, magnetic resonance, angioscopy, color flow Doppler, cystoscopy, diaphanography, echocardiography, fluoresosin angiography, laparoscopy, magnetic resonance angiography, positron emission tomography, single-photon emission computed tomography, x-ray angiography, computed tomography, nuclear medicine, biomagnetic imaging, culposcopy, duplex Doppler, digital microscopy, endoscopy, fundoscopy, laser surface scan, magnetic resonance spectroscopy, radiographic imaging, thermography, and radio fluroscopy. Other types of imaging modalities can be used, and the claims should not be limited to a specific type of imaging modality or medical diagnostic imaging system unless explicitly recited therein.

It is intended that the foregoing detailed description be understood as an illustration of selected forms that the invention can take and not as a definition of the invention. It is only the following claims, including all equivalents, that are intended to define the scope of this invention.

What is claimed is:

1. A method for migrating presets between medical diagnostic imaging system platforms, the method comprising:
(a) providing a preset for a first medical diagnostic imaging system platform to a preset translator;
(b) with the preset translator, translating the preset for the first medical diagnostic imaging system platform to a second medical diagnostic imaging system platform; and
(c) implementing the translated preset on a medical diagnostic imaging system comprising the second medical diagnostic imaging system platform.

2. The method of claim 1, wherein the preset translator is located in the medical diagnostic imaging system comprising the second medical diagnostic imaging system platform.

3. The method of claim 2, wherein (a) comprises sending the preset for the first medical diagnostic imaging system platform from a medical diagnostic imaging system comprising the first medical diagnostic imaging system platform to the medical diagnostic imaging system comprising the second medical diagnostic imaging system platform.

4. The method of claim 1, wherein the preset translator is located in a medical diagnostic imaging system comprising the first medical diagnostic imaging system platform.

5. The method of claim 4 further comprising sending the translated preset from the medical diagnostic imaging system comprising the first medical diagnostic imaging system platform to the medical diagnostic imaging system comprising the second medical diagnostic imaging system platform.

6. The method of claim 1, wherein the preset translator is located in a server in communication with the medical diagnostic imaging system comprising the second medical diagnostic imaging system platform.

7. The method of claim 6, wherein (a) comprises sending the preset for the first medical diagnostic imaging system platform from a medical diagnostic imaging system comprising the first medical diagnostic imaging system platform to the preset translator in the server.

8. The method of claim 6, wherein (a) comprises sending the preset for the first medical diagnostic imaging system platform from a preset library in the server to the preset translator in the server.

9. The method of claim 6 further comprising sending the translated preset from the server to the medical diagnostic imaging system comprising the second medical diagnostic imaging system platform.

10. The method of claim 1 further comprising presenting the translated preset for user review.

11. The method of claim 1, wherein the preset for the first medical diagnostic imaging system platform comprises a plurality of items, and wherein (b) comprises consulting a conversion table for each respective item.

12. The method of claim 11, wherein if an item is found in the conversion table, performing one of the following: modifying the item, deleting the item, adding a new item based on the item, and performing no modification to the item.

13. The method of claim 11, wherein if an item is not found in the conversion table, allowing a user to perform one of the following: a manual translation and a guided translation.

14. A system for sharing medical diagnostic imaging system presets, the system comprising:
a server;
a first medical diagnostic imaging system in communication with the server, the first medical diagnostic imaging system operative to send an medical diagnostic imaging system preset to the server; and
a second medical diagnostic imaging system in communication with the server, the second medical diagnostic imaging system operative to request the medical diagnostic imaging system preset from the server;
wherein the server is operative to send the requested medical diagnostic imaging system preset to the second medical diagnostic imaging system in response to the request from the second medical diagnostic imaging system.

15. The system of claim 14, wherein the requested medical diagnostic imaging system preset is for a first medical diagnostic imaging system platform, wherein the second medical diagnostic imaging system comprises a second medical diagnostic imaging system platform, and wherein the server comprises a preset translator operative to translate the requested preset to the second medical diagnostic imaging system platform.

16. The system of claim 14, wherein the requested medical diagnostic imaging system preset is for a first medical diagnostic imaging system platform, wherein the second medical diagnostic imaging system comprises a second medical diagnostic imaging system platform, and wherein the second medical diagnostic imaging system comprises a preset translator operative to translate the requested preset to the second medical diagnostic imaging system platform.

17. A method for sharing medical diagnostic imaging system presets, the method comprising:
(a) providing a server and first and second medical diagnostic imaging systems in communication with the server;
(b) sending a medical diagnostic imaging system preset from the first medical diagnostic imaging system to the server;
(c) storing the medical diagnostic imaging system preset in the server;
(d) sending a request to the server for the medical diagnostic imaging system preset; and
(e) in response to the request, sending the requested medical diagnostic imaging system preset from the server to the second medical diagnostic imaging system.

18. The method of claim 17, wherein the preset is established by a user, and wherein the request for the preset in (d) is made by a different user.

19. The method of claim 17, wherein the preset is established by a user, and wherein the request for the preset in (d) is made by the same user.

20. The method of claim 17, wherein the request is sent from the second medical diagnostic imaging system.

21. The method of claim 17, wherein the requested medical diagnostic imaging system preset is for a first medical diagnostic imaging system platform, wherein the second medical diagnostic imaging system comprises a second medical diagnostic imaging system platform, and wherein the method further comprises translating the requested preset to the second medical diagnostic imaging system platform.

22. The method of claim 21, wherein the translation is performed by the server.

23. The method of claim 21, wherein the translation is performed by the second medical diagnostic imaging system.

24. The method of claim 1, wherein the preset for the first medical diagnostic imaging system platform is provided to the preset translator in a common format.

25. The method of claim 24, wherein a medical diagnostic imaging system comprising the first medical diagnostic imaging system platform places the preset for the first medical diagnostic imaging system platform in the common format.

26. The method of claim 25 further comprising sending the preset for the first medical diagnostic imaging system platform in the common format from the medical diagnostic imaging system comprising the first medical diagnostic imaging system platform to a medical diagnostic imaging system comprising the second medical diagnostic imaging system platform, wherein the preset translator is located in the medical diagnostic imaging system comprising the second medical diagnostic imaging system platform.

27. The method of claim 25 further comprising:
sending the preset for the first medical diagnostic imaging system platform in the common format from the medical diagnostic imaging system comprising the first medical diagnostic imaging system platform to a server; and
sending the preset for the first medical diagnostic imaging system platform in the common format from the server to a medical diagnostic imaging system comprising the second medical diagnostic imaging system platform;
wherein the preset translator is located in the medical diagnostic imaging system comprising the second medical diagnostic imaging system platform.

28. The method of claim 25 further comprising:
sending the preset for the first medical diagnostic imaging system platform in the common format from the medical diagnostic imaging system comprising the first medical diagnostic imaging system platform to a server; wherein the preset translator is located in the server; and
sending the preset for the second medical diagnostic imaging system platform from the server to a medical diagnostic imaging system comprising the second medical diagnostic imaging system platform.

29. The method of claim 24 further comprising:
sending the preset for the first medical diagnostic imaging system platform from a medical diagnostic imaging system comprising the first medical diagnostic imaging system platform to a server; wherein the server places the preset for the first medical diagnostic imaging system platform in the common format; and
sending the preset for the first medical diagnostic imaging system platform in the common format from the server to a medical diagnostic imaging system comprising the second medical diagnostic imaging system platform; wherein the preset translator is located in the medical diagnostic imaging system comprising the second medical diagnostic imaging system platform.

30. The method of claim 1, wherein the medical diagnostic imaging system comprises an ultrasound imaging system.

31. The system of claim 15, wherein the first medical diagnostic imaging system is operative to send the medical diagnostic imaging system preset to the server in a common format.

32. The system of claim 16, wherein the first medical diagnostic imaging system is operative to send the medical diagnostic imaging system preset to the server in a common format, and wherein the server sends the medical diagnostic imaging system preset to the second medical diagnostic imaging system in a common format.

33. The system of claim 16, wherein the server is operative to send the medical diagnostic imaging system preset to the second medical diagnostic imaging system in a common format.

34. The system of claim 14, wherein the medical diagnostic imaging system comprises an ultrasound imaging system.

35. The method of claim 21, wherein the first medical diagnostic imaging system places the preset in a common format, and wherein the medical diagnostic imaging system preset is sent from the first medical diagnostic imaging system to the server in a common format.

36. The method of claim 21, wherein the server places the preset in a common format, and wherein the medical diagnostic imaging system preset is sent from the server to the second medical diagnostic imaging system in the common format.

37. The method of claim 17, wherein the medical diagnostic imaging system comprises an ultrasound imaging system.
